(19) United States
(12) Patent Application Publication
(10) Pub. No.: US 2020/0406032 A1
(43) Pub. Date: Dec. 31, 2020

(54) PACEMAKER NETWORK

(71) Applicants: Mehnert WALTER, Ottobrunn (DE); Thomas Theil, Feldafing (DE)

(72) Inventors: Mehnert WALTER, Ottobrunn (DE); Thomas Theil, Feldafing (DE)

(21) Appl. No.: 16/914,978

(22) Filed: Jun. 29, 2020

(30) Foreign Application Priority Data
Jun. 28, 2019 (DE) 102019117586.0
Jul. 15, 2019 (DE) 102019119117.3
Sep. 2, 2019 (DE) 102019123464.6
Sep. 11, 2019 (DE) 102019124435.8

(51) Int. Cl. A61N 1/365; A61N 1/375

(52) U.S. Cl. CPC A61N 1/365; A61N 1/37512

(57) ABSTRACT

A wireless pacemaker network including an electronic pacemaker unit (1) that acts as a master in the pacemaker network, including an electrode portion (2) that is, according to its intended purpose, to be attached to a first body portion, and an electronics assembly; and an electronic pacemaker unit (1′) that acts as a slave in the pacemaker network. The electronic pacemaker unit (1′) including an electrode portion (2′) that is, according to its intended purpose, to be attached to a second body portion, and an electronics assembly connected with the electrode portion, which is configured to generate a pulse, in particular a voltage pulse, and to output it to the second body portion via the electrode portion; wherein the pacemaker unit (1) acting as the master and the pacemaker unit (1′) acting as the slave interact wirelessly for controlling the bodily function.

The invention relates to an electronic pacemaker network for implantation in a body of a living being and for controlling a bodily function.

Pacemakers are generally known in the prior art and are used for various different medical indications.

Pacemakers are used, for example, as so-called dual-chamber cardiac pacemakers for human heart diseases.

The essential components of such a dual-chamber pacemaker form cable electrodes fixed to a housing, which are connected to the different chambers of the diseased heart of the patient, and an electronics assembly accommodated in the housing, which can monitor functions of the chambers via the electrodes and deliver stimulation pulses to the chambers when determining a dysfunction. All mentioned components are connected by cable and are, nowadays, fully implanted in the human body.

In general, the housing is arranged in the vicinity of the heart, for example, below the sternum or clavicle, and the cable electrodes are guided to the heart chambers to be stimulated. The cable electrodes used for this purpose are designed relatively long, in order to reach the heart chambers within the body of the patient.

There are limits to a desired miniaturization of the known dual-chamber pacemakers in such a structure.

In addition, the cable electrodes are vulnerabilities, because necessary insulations may cause rejection reactions of the body of the patient and may require replacement of the cable electrodes after a certain period of time due to occurring aging in particular by mechanical friction or deformation.

Against the above background, it is an object of the present invention to provide opportunities to stimulate body portions—being separated from each other by a distance—and to allow decreasing an electronic structure necessary for this purpose.

This object is achieved by a pacemaker network according to claim 1. Preferred embodiments are the subject-matters of the dependent claims.

The basic idea of the invention is to form pacemakers which have to stimulate different body portions, in that individual pacemaker units associated with respective body portions are connected wirelessly with each other in a pacemaker network. The structure of a wireless pacemaker network allows for rendering electrode portions necessary for the stimulation very short and small, respectively, and for rendering a correspondingly associated electronics assembly smaller.

In accordance with the above basic idea of the invention, a wireless pacemaker network for implantation in a body of a living being and for controlling a bodily function comprises:

an electronic pacemaker unit that acts as a master in the pacemaker network, comprising
an electrode portion that is, according to its Intended purpose, to be attached to/arranged at a first body portion, and
an electronics assembly that is adapted to monitor a function or own activity of the first body portion preferable via the electrode portion and/or to generate a pulse, in particular a voltage pulse, and to output it via the electrode portion to the first body portion; and

an electronic pacemaker unit that acts as a slave in the pacemaker network, comprising
an electrode portion that is, according to its intended purpose, to be attached to/arranged at a second body portion, and
an electronics assembly connected with the electrode portion, which is configured to generate a pulse, in particular a voltage pulse, and to output it to the second body portion via the electrode portion; wherein

the pacemaker unit acting as the master and the pacemaker unit acting as the slave are configured to interact wirelessly with each other for controlling the bodily function, by the electronics assembly of the pacemaker unit acting as the slave (i) to obtain information on the function or own activity of the first body portion from the pacemaker unit acting as the master and/or on the delivery of the pulse to the first body portion, and (ii) to determine, based on the information, whether and/or when the pulse is delivered to the second body portion.

Preferably, the electronics assembly of the pacemaker unit acting as the slave may be configured—as the one of the pacemaker unit acting as the master—to monitor a function or own activity of the second body portion, preferably via the electrode portion or a separate monitoring electrode, and/or to generate the pulse, in particular the voltage pulse, and to deliver it to the second body portion via the electrode portion.

In a preferred embodiment to which the invention is not limited, the pacemaker network according to the invention takes over the function of a multi-chamber cardiac pacemaker, for example, a dual-chamber cardiac pacemaker.

That is, the control taken by the pacemaker network of the invention, for example, relates to the control of the human heart beat by monitoring the individual chambers of the heart and/or stimulating them by pulses.

Therefore, the pacemaker unit acting as the master is intentionally associated, for example, to an atrium (Atrium) of the human heart, which corresponds to the first body portion, and the pacemaker unit acting as the slave is intentionally associated to of a main chamber (Ventricle) of the human heart, which corresponds to the second body portion.

The association is done in particular in that the electrode portions of the pacemaker unit acting as the master and of the pacemaker unit acting as the slave are anchored at the respective first and second body portions.

The anchoring, for example, is done by a wire spiral that is rotated, when implanted, into a tissue of the human heart. Alternatively, barbs can provide for the anchoring, which interlock when penetrating the tissue. Further alternatively, the electrode portion of the pacemaker unit acting as the master and of the pacemaker unit acting as the slave may be formed as a flat electrode, which is exposed on an outer surface of a casing/sleeve explained in the following, of the respective pacemaker unit.

If the pacemaker network of the invention, beyond this extent, shall provide for the stimulation of a further chamber of the heart, that is the pacemaker network of the invention functions as a triple-chamber pacemaker, a further pacemaker unit acting as a slave is provided in the pacemaker network, which is anchored at a third body portion via a corresponding electrode portion.

Said pacemaker units interact together in a wireless manner, which is why they can respectively be arranged extremely close to the corresponding body portions, and the electrode portions can be formed very small and, preferably, without a corresponding plastic insulation.

Preferably, the pacemaker network of the invention is formed in that the pacemaker unit acting as the master comprises a transmitting unit and is configured to transmit the information via the transmitting unit, and that the pacemaker unit acting as the slave comprises a receiving unit and is configured to obtain the information by receiving the information transmitted by the transmitting unit via the receiving unit.

The transmitting unit can base on various wireless technologies, such as Bluetooth, In particular Low Energy Bluetooth, or, in general, on wireless technologies with very high or very low radio frequencies, the latter being preferred.

Further preferably, the pacemaker network of the invention may be formed so that the pacemaker unit acting as the slave has a detection unit and is configured to obtain the information by detecting the pulse delivered by the pacemaker unit acting as the master, via the detection unit.

The pacemaker unit acting as the master requires in particular no transmitting unit in this case.

If the pacemaker network of the invention functions as a dual-chamber pacemaker, for example, by means of the above described configuration of the pacemaker network of the invention, the following functionality can be realized by the pacemaker network:
The electronics assembly of the pacemaker unit acting as the master and associated to the atrium (Atrium) and the first body portion, respectively, monitors the function or own activity of the atrium, preferably via its electrode portion or via the separate monitoring electrode.
If the electronics assembly, in the context of monitoring of the first body portion, determines a corresponding own activity, it communicates this information, for example via the transmitting unit to the pacemaker unit acting as the slave, which receives the corresponding information, for example via its receiving unit.
The pacemaker unit acting as the slave decides, based on the received information, when to deliver the pulse to the chamber (ventricle) and the second body portion, respectively, via its corresponding electrode portion. This takes place for example after receiving the information in that the electronics assembly of the pacemaker unit acting the slave always generates the pulse after expiration of predetermined period (A/V-interval), independently of an own activity of the second body portion, and delivers the pulse, for stimulation, to the second body portion via its electrode portion.

In general, as already explained, the pacemaker unit acting as the slave preferably may be configured to monitor a function and own activity of the second body portion, respectively, preferably via its electrode portion or the separate monitoring electrode.

A preferred variation of the function mentioned above results from this preferred configuration of the pacemaker unit acting as the slave in that the electronics assembly of the pacemaker unit acting as the slave generates the pulse and delivers it to the second body portion via its electrode portion, only if, in the course of monitoring the second body portion, no function and own activity of the second body portion, respectively, is detected by the electronics assembly until expiration of said period (A/V-interval). I.e. The electronics assembly of the pacemaker unit acting as the slave decides whether and, if yes, when the pulse is to be delivered.

In the framework of the described operational mode of the pacemaker network of the invention, the electronics assembly of the pacemaker unit acting as the master is configured to monitor the functions and own activity of the first body portion, respectively. The described monitoring function may be sufficient for certain medical indications, for example, in the case that the function and own activity of the atrium is not disturbed and does not need stimulation. In other words, the pacemaker unit acting as the master can perform only the function of monitoring the first body portion.

The opposite case that the electronics assembly of the pacemaker unit acting as the master does not comprise any monitoring function, but is configured to always generate the pulse and to deliver it to the first body portion is also a conceivable preferred variant. For example, a dual chamber pacemaker may be realized by the pacemaker network of the invention, having the following operational mode:
The electronics assembly of the pacemaker unit acting as the master and associated to the atrium (Atrium) and the first body portion, respectively, always generates the pulse and delivers it to the first body portion via its corresponding electrode portion.
The pacemaker unit acting as the master transmits this information, for example via the transmitting unit, to the pacemaker unit acting as the slave which receives the corresponding information for example via its receiving unit. In the alternative, the pacemaker unit acting as the slave preferably may also obtain the information via its detection unit; for this case, the pacemaker unit acting as the master requires no transmitting unit.
The pacemaker unit acting as the slave decides, based on the received/obtained information on delivering the pulse to the chamber (ventricle) and the second body portion, respectively. This may take place, as described above, either independently, i.e. without monitoring the own activity of the second body portion, or dependently, i.e. with monitoring the own activity of the second body portion.

Ultimately, the combined case is conceivable in the context of the invention, that the electronics assembly of the pacemaker unit acting as the master has both functions, i.e. to monitor the first body portion and to generate/deliver the pulse when needed. For example, a dual chamber pacemaker may be realized by the pacemaker network of the invention, having the following operational mode:
The electronics assembly of the pacemaker unit acting as the master and associated to the atrium (Atrium) and the first body portion, respectively, monitors the function or own activity of the atrium, preferably via its electrode portion or via the separate monitoring electrode. In case the electronics assembly does not detect any corresponding own activity in context of monitoring the first body portion, it generates the corresponding pulse and delivers it to the first body portion via its electrode portion.
The electronics assembly transmits then either the information that monitoring revealed an own activity and function of the first body portion, respectively, or the information that the pulse was delivered to the first body portion, to the pacemaker unit acting as the slave, for example, via the already described transmitting unit.
The pacemaker unit acting as the slave decides, based on the received information, on delivering the pulse to the chamber (ventricle) and the second body portion, respectively. This may take place, as described above, either independently, i.e. without monitoring the own activity of the second body portion, or dependently, i.e. with monitoring the own activity of the second body portion.

The pacemaker unit acting as the master and/or the pacemaker unit acting as the slave which correspond to the core idea of the pacemaker network of the invention, preferably comprise(s):

an energy storage, for example an accumulator or a capacitor (for example a gold cap) to supply the electronics assembly with electrical energy, which can be recharged with electrical energy after discharge.

The type of energy storage may be chosen at will. The energy storage may, for example, be an accumulator, preferably a lithium-on accumulator. Alternatively, the energy storage may be a capacitor with preferably low self-discharge. The energy storage may preferably be hermetically encapsulated so that it does not present any danger to the body of the living being.

Preferably, the pacemaker unit acting as the master and/or the pacemaker unit acting as the slave of the pacemaker network which corresponds to the core idea of the invention, respectively comprise:

a charging impulse generation portion electrically connected to the energy storage, which is configured to emit a charging impulse to the energy storage for the recharging of the energy storage, wherein the charging impulse generation portion comprises a magnetization portion with oriented magnetic domains, which can be contactlessly influenced by a changing (external) magnetic field so that, when a certain field strength (amplitude) is reached, a remagnetization wave, caused by the continuously reversing magnetic domains, occurs in the magnetization portion which runs across the magnetization portion and leads to the generation of the charging impulse.

The mentioned external or externally generated magnetic field is preferably generated by the charging device according to the invention, which will be explained below, for wirelessly recharging the energy storage.

The magnetization portion of the charging impulse generation portion comprises equally oriented magnetic domains which, together, can be influenced by the changing externally generated magnetic field. When the externally generated magnetic field reaches, in a certain area of the magnetization portion, a certain amplitude or field strength that is in the range of a few millitesla (less than or equal to 10 mT), the domains reverse their magnetization polarity in this area (reversal of the so-called Weiss domains), whereby said remagnetization wave starts running across the magnetization portion, as it is the case for example in a Wiegand or impulse wire still to be mentioned below.

The form of the magnetization portion is arbitrary.

When the strength of the externally generated magnetic field reaches, for example at one end of the magnetization portion, the certain amplitude or strength, the remagnetization wave begins to run at this end of the magnetization portion until it reaches the other end of the magnetization portion. From a physical perspective, this thus occurring remagnetization wave is essentially a Bloch wall that runs across the magnetization portion.

The remagnetization of the magnetization portion is used to generate the charging impulse, for example by induction.

It should be specifically mentioned here that the size (amplitude) and the speed of the remagnetization wave does not depend, or only insignificantly, on the frequency of the changing externally generated magnetic field, but primarily on the material data of the magnetization portion. The trigger point of the remagnetization wave depends on the circumstance when the changing externally generated magnetic field reaches the mentioned amplitude or field strength, wherein the gradient of the change of the magnetic field and the corresponding frequency, respectively, does not play a role. When the necessary strength (amplitude) of the magnetic field is reached, the Bloch wall and the remagnetization wave, respectively, begins to run.

The commutation frequency or the change of the magnetic field only play a role, albeit a subordinate role, because it only provides information about the number of the initiations of the remagnetization wave or it only indicates, respectively, how often the remagnetization wave is initiated and therefore how often a charging impulse is generated.

It is particularly preferred that the pacemaker unit acting as the master and the pacemaker unit acting as the slave, particularly preferred all pacemaker units of the pacemaker network, have the charging impulse generation portion and thus their respective energy storages can be recharged without contact. However, it is alternatively conceivable that either only the pacemaker unit acting as the master or only the pacemaker unit acting as the slave has the charging impulse generation portion, for example in the explained case that the pacemaker unit acting as the master does not have to generate any pulses and can be operated with very low energy.

The above statements apply equally to the following preferred configurations.

Preferably, the charging impulse generation portion of the respective pacemaker unit(s) has at least one coil which is spatially arranged to the magnetization portion in such a way that it generates, when the remagnetization wave occurs, a voltage pulse leading to the charging impulse.

The spatial arrangement may be such that the coil is wound around the magnetization portion, and in particularly that it axially surrounds the same.

It is common knowledge that coils made from electrically conductive materials are inductances. The remagnetization wave causes the coil, due to its inductive properties, to generate the voltage impulse that leads to the charging impulse.

For each commutation of the changing magnetic field, the coil therefore generates a voltage impulse of a certain strength (Independently from how quickly or with which frequency the magnetic field changes). The magnetization portion and the coil may, for example, be dimensioned so that the amplitude of the voltage impulse that leads to the charging impulse is 10V or higher.

The voltage impulses generated by the coil have alternating reversed polarities. To use all voltage impulses, the charging impulse generation portion comprises a charging electronics that preferably rectify the voltage impulses by means of a rectifier and/or temporarily store them in a capacitor.

In general terms, the advantage is that a part of the magnetic energy of the changing magnetic field first accumulates in the magnetization portion and is then quite suddenly released in the form of the moving remagnetization wave. The induction and therefore the creation of the electrical voltage consequently occurs on/in the col at this point in time at first.

This means that the contactless energy transmission is not based on the fact that the changing magnetic field is used immediately for the voltage induction in the coil, but that the corresponding energy of the magnetic field is temporarily stored in the magnetization portion and then released quite suddenly when the remagnetization wave is initiated. For this reason, the commutation frequency may be adapted so that energy can be transmitted through a casing or sleeve made from metal without any problems. It is an indirect induction method; i.e., the change of the magnetic flux generated by the electricity of the primary coil does not exclusively lead to voltage in the secondary col, as is the case with the direct induction, but part of this flux is initially temporarily stored in the magnetization portion. At a certain field strength, the flux then stimulates the magnetization portion to generate a magnetic impulse wave of a certain polarity (remagnetization wave) and therefore indirectly in the secondary coil a voltage impulse of a certain polarity with a significantly higher amplitude. The coil of the charging impulse generation portion acts here as said secondary coil that generates the voltage impulse when the remagnetization wave occurs. Said primary coil is located for example in the charging device to be explained below.

In this point, there are significant differences from known contactless charging processes for accumulators which use an electromagnetic alternating field for the transmission of energy. Such an electromagnetic alternating field with a high frequency can hardly or at least very difficultly be used in pacemakers to be implanted because the penetration depth of the electromagnetic alternating field into the body of the living being, particularly into a metal casing, is too low due to the effects that occur such as, for example, the skin effect.

The explained charging impulse generation portion, which is contained in at least one of the pacemaker units, but preferably in the pacemaker unit acting as the master and the pacemaker unit acting as the slave, contributes greatly to the fact that the pacemaker units and thus the entire pacemaker network can be made smaller.

The background for this is the fact that the energy storages of the corresponding pacemaker units can be recharged contactlessly, without being confronted with the problems mentioned above, which occur during high-frequency contactless recharging which utilizes direct induction.

This creates space to greatly reduce the energy storages and to design/construct the respective electronics assembly of the pacemaker units with only secondary consideration of their power consumption. For example, the energy storages and/or a power consumption of the electronics assembly or units (transmitting unit, receiving unit, detection unit) could be designed/dimensioned in such a way that the energy content of the energy storages last only for a few months, for example 6 to 12 months, or years, for example 1 or 2 years.

Such power consumption or short running time would be a KO criterion for known pacemakers whose power supply is ensured by a normal battery, because surgical interventions necessary for battery replacement would have an unacceptably high frequency.

The magnetization portion is preferably configured, due to a special, e.g. mechanical, machining, so that the magnetic domains of the magnetization portion are equally oriented.

The magnetization portion preferably comprises a magnetically hard shell area which encloses a magnetically soft core area.

The magnetically hard shell is created for example when the magnetization portion is machined and produced. A preferred material for the magnetization portion is vicalloy, which is machined for example in cold forming steps to orient the magnetic domains.

Preferably, the magnetization portion is at least an impulse wire or a Wiegand wire. The magnetization portion may also comprise a plurality of impulse wires or a plurality of Wiegand wires or a combination of at least one impulse wire and one Wiegand wire.

The number of the coils is not limited to a single coil as well. Each of the wires may be assigned a coil of its own, or, alternatively, a plurality of the wires may be surrounded by one or more coils. Preferably, the coil is, or the coils are, wound around one or more of the wires.

In this case, the coils each form a secondary coil of the indirect induction method explained above, to which energy is indirectly transmitted via the magnetization portion from the primary coil, which is preferably located in the charging device still to be explained.

Preferably, the pacemaker network is configured so that the electronics assembly of the pacemaker unit acting as the master and/or of the pacemaker unit acting as the slave are fully surrounded, together with the respective energy storage and the respective charging impulse generation portion, by a sleeve or a casing which is formed of a material that is not rejected by the body of the living being. The material is preferably a non-ferromagnetic metal, in particular titanium, or a metal alloy comprising titanium in particular.

Alternative metals are stainless steels.

The electric conductivity of these materials is important to dampen high-frequency interfering fields that may impair the function of the pacemaker network, for example having the functions of a dual-chamber pacemaker. The electrically conductive casing can also be provided as a mass contact for the current circuit. If the casing is made of a non-conductive material, a separate mass electrode may be provided.

For example, the electrode portion is detachably attached to and passes through the sleeve/casing, wherein the electrode portion is preferably insulated from the sleeve/casing, and is connected to the electronics assembly inside the sleeve/casing. Alternatively, the electrode portion can be formed by electrode surfaces exposed on an outer surface, that touch the corresponding body portion.

The outer shape of the pacemaker unit(s) may be such that the casing accommodates said elements, and that the electrode portion projects or is exposed on one side of the casing. The functionality of contactless recharging allows such a strong reduction of the casing and the electrode portion that the corresponding pacemaker unit can potentially also be pushed into a human heart, i.e. Into an interior of the atrium or chamber, and anchored there to the corresponding body portion or make contact there with the body portion, via its electrode portion.

For example, a preferred volume of the casing is of the order of less than 1, 2, 3, 4, 5, 6, 7 or 8 cm³; for example, the casing has a spherical shape with 1 cm diameter. Alternatively, the casing can be cylindrical in shape with a diameter of 1 or 0.5 cm, for example, and a height of 2.5 cm, for example. The length of the electrode portion can be of a few centimeters, i.e. less than 1 cm, i.e. a few millimeters. Preferably, the electrode portion has no plastic insulation and, if implanted as intended, is almost completely anchored in the corresponding body portion in such a way that the casing touches the body portion.

The fact that was already mentioned, that the magnetization portion accumulates the magnetic energy and only its release in the form of the remagnetization wave leads to the generation of the induced electrical voltage and the voltage impulse, respectively, within the casing and the sleeve, respectively, creates freedom for the selection of the preferably non-ferromagnetic material of said sleeve and casing, respectively, because no direct requirements as to the transmission frequency must be met.

Preferably, the charging impulse generation portion of the pacemaker unit(s) comprises, in a direction in which the at least one coil is wound or in which the coils are wound, a magnetic collecting lens at at least one end portion of the magnetization portion for bundling and guidance of the changing externally generated magnetic field to the magnetization portion.

Said direction corresponds to the longitudinal direction of the coil or the coils in which it is wound/they are wound. Preferably, at least one magnetic collecting lens is arranged at both end portions of the magnetization portion, which bundle the changing externally generated magnetic field on the magnetization portion and/or lead it to the same.

Alternatively to the use of an independent collecting lens/collecting lenses, there is also the possibility that the sleeve and the casing, respectively, of the pacemaker unit acting as the master and/or the pacemaker unit acting as the slave is partially or sectionally formed from ferromagnetic material or is partially or sectionally coated with such a material and, for example by a separation into two separate halves, is configured to directly take up the function of the magnetic collecting lenses. Due to the then larger configuration of the collecting lenses, the magnetic field to be generated by the charging device can then be reduced further.

The at least one magnetic collecting lens of the pacemaker unit(s) is preferably formed from ferromagnetic material which bundles the externally generated magnetic field for the magnetization portion.

The magnetic collecting lens(es) is/are made for example from ferrite and have, for example, the form of a hollow cylinder, the axis of which points in the direction of the respective end portion of the magnetization portion.

The magnetization portion is preferably inserted into the hollow cylinder.

The use of the magnetic collecting lens(es) makes it possible, for example, that the charging device, which will be explained below, can generate a weaker magnetic field and that its orientation is less critical in relation to the pacemaker unit(s).

The electronics assembly of the pacemaker unit acting as the master and/or of the pacemaker unit acting as the slave preferably does not comprise any elements from ferromagnetic materials, and/or the charging impulse generation portion of the electronic pacemaker according to the invention does not comprise any elements from ferromagnetic materials except for the magnetization portion, and, when preferably provided, the at least one collecting lens.

This configuration of the pacemaker unit acting as the master and/or of the pacemaker unit acting as the slave is advantageous in that the elements formed from non-ferromagnetic materials are not impaired or interfered with by the externally generated magnetic field.

Further, the electronics assembly of pacemaker unit acting as the master and/or of the pacemaker unit acting as the slave is configured to transmit a signal indicative of the quality of the charging impulse.

Said signal may, for example, be a low-frequency signal that penetrates the body of the living being and, if no respective antenna is provided, the sleeve or the casing of the pacemaker according to the invention.

If the pacemaker unit acting as the master has the explained transmits ting unit, the signal indicating the quality can be transmitted by the transmitting unit.

The quality of the charging impulse is proportional to the value of the integral (∫ i dt). With good charging impulses, i.e. with very high quality, the value is for example 100 nC.

The quality of the charging impulses can be derived, for example, from how strongly successive charging impulses fluctuate when the external magnetic field changes, i.e. how their current and/or voltage amplitudes fluctuate and/or how their time widths vary.

Furthermore, for example, the signal indicating the quality can also indicate the value of the integral of the current of the charging impulse over time (∫ i dt), i.e. Its charge content.

In the alternative, the signal indicating the quality of the charging impulse may be a binary signal, for example, which assumes an OK state when the charging impulse and its charge content, respectively, exceeds a threshold and an NG state when the charging impulse does not exceed the threshold. For example, the threshold value can be 50%, 55%, 60%, 65%, 70%, 75%, 80%, 85% or 90% of the charge content deliverable by the magnetization portion. As an example, values of 50 nC, 55 nC, 60 nC, 65 nC, 70 nC, 75 nC, 80 nC, 85 nC or 90 nC can be given.

The electronics assembly can be set up in such a way that it transmits the signal indicating the quality for each charging impulse or alternatively only for those charging impulses that occur at certain intervals in succession. If the external magnetic field is generated by a charging device, which is explained in more detail below, which generates the magnetic field with a commutation frequency in the kHz range, the electronics assembly can generate the signal indicating the quality, for example for those of the charging impulses which occur at intervals of, for example, >1, 25, 50, 100, 200, 500, 750, or 1000 charging pulses.

The transmitting unit of the pacemaker unit acting as the master and the receiving unit of the pacemaker unit acting as the slave, both described above, can both preferably have transmitting and receiving functions, and thus can serve, for example, as an interface for programming the pacemaker unit(s). In this context, the pacemaker units can be reprogrammed thereby in such a way that they work completely autonomously from each other, i.e. each pacemaker unit monitors the own activity of the corresponding body portion independently and generates the impulse independently as needed. Furthermore, the pacemaker units could thereby be switched on or off.

In addition, the corresponding electronics assembly can be configured in such a way that a charge state of the corresponding energy stores can be queried via the transmitting and receiving function of the pacemaker unit acting as the master and/or the pacemaker unit acting as the slave.

As an alternative to the signal indicating the quality, the charging device described below can be configured in such a way that it queries, by means of the mentioned functions, the charge state of the energy storage(s) at predetermined time intervals and draws a conclusion on the quality of the individual charging impulses on the basis of the information about a change in the charge state(s), the time interval and the commutation frequency mentioned below. The time interval is preferably 0.5 min, 1.0 min, 1.5 min, 2.0 min, 2.5 min, 3.0 min, 3.5 min, 4.0 min, 4.5 min, 5.0 min.

The invention also relates to a charging device for a pacemaker network, wherein the charging device is configured to generate a magnetic field that changes with a commutation frequency and amplitude. The charging device, when used as intended, is arranged on a body surface of the living being or close to the body surface of the living being so that the magnetic field penetrates the body and the implanted pacemaker unit acting as the master and/or the pacemaker unit acting as the slave to influence the corresponding charging impulse generation portion.

The commutation frequency preferably ranges from
X to 10 kHz, wherein X>0 and X>=0.1 kHz, 0.2 kHz, 0.3 kHz, . . . , 4.9 kHz, . . . , 9.9 kHz.

The charging device comprises for example one or a plurality of coils that act in the indirect induction method referred to above as the primary coil(s). It is preferred that a core, for example made from ferrite, is inserted into the coil(s).

As intended, the charging device generates a flow of electric current through the coil(s) to generate a changing electromagnetic field that commutes with the mentioned commutation frequency. When the charging device is arranged on the body surface and in its vicinity, respectively, the alternating field can penetrate the body of the living being and the pacemaker unit(s).

The magnetic portion of the changing electromagnetic field forms the externally generated magnetic field explained above, which influences the magnetization portion for the initiation of the remagnetization wave.

The strength and/or the commutation frequency of the electromagnetic alternating field could preferably be controlled in the charging device. This is preferable in that the charging device may be set depending on the location of the pacemaker units within the body and on the necessary penetration depth, respectively.

Preferably, the charging device according to the invention comprises a plurality of coils for the generation of the changing magnetic field, wherein the plurality of coils can be controlled accordingly for an optimization of the charging impulse(s), on the basis of the signals indicating the quality of the charging impulse(s), for example on the basis of their absolute values and/or their change.

Alternatively, the charging device according to the invention includes a plurality of coils for generating the changing magnetic field, wherein

the charging device is configured (i) to query the charge state of the energy storage of the pacemaker unit acting as the master and/or the pacemaker unit acting as the slave at specific time intervals and to draw a conclusion on the quality of the charging impulses on the basis of the change in the charge state(s), the time interval and the commutation frequency, and (ii) to control the plurality of coils accordingly on the basis of the drawn conclusion.

The coils of the plurality of coils are preferably spatially arranged so that the charging device can adjust the orientation of the generated magnetic field by controlling the coils. This has the advantage that the charging device can change the orientation of the magnetic field taking into account the signals (e.g. their absolute values and/or their change) Indicating the quality of the charging impulse(s) or taking into account the drawn conclusion, in order to improve or optimise the quality of the charging impulses.

The charging device is preferably configured to automatically control the coils for orienting of the magnetic field.

Below, a preferred embodiment of the invention is explained with reference to the figure.

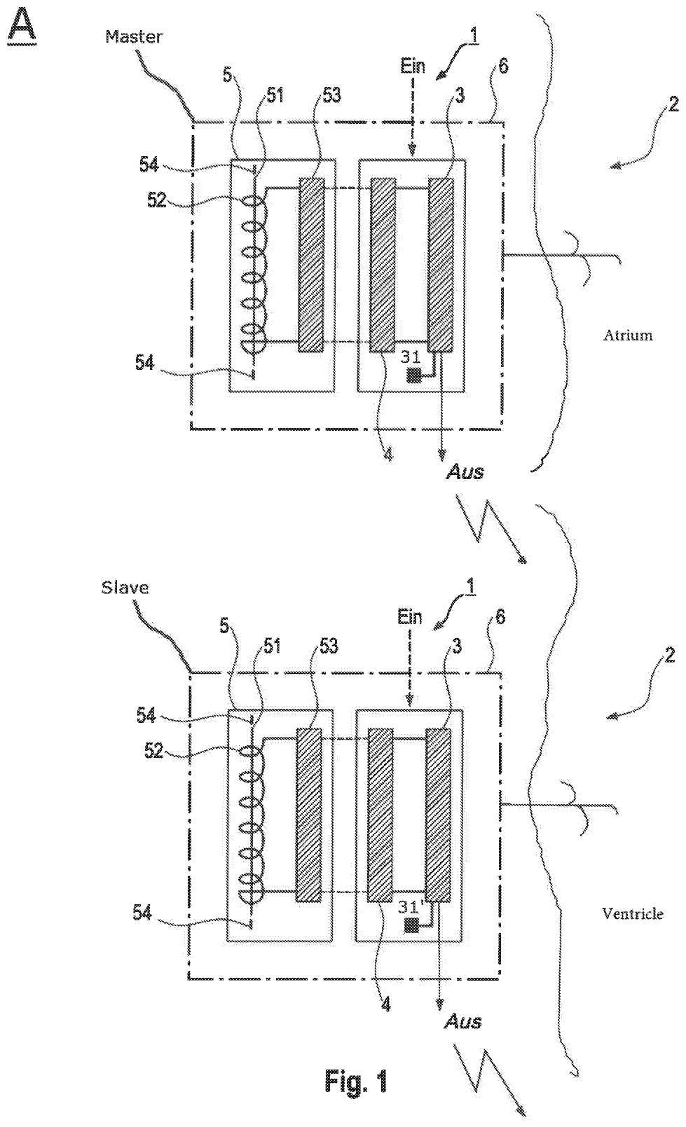

FIG. 1 shows a schematic illustration of an implant pacemaker according to the invention;

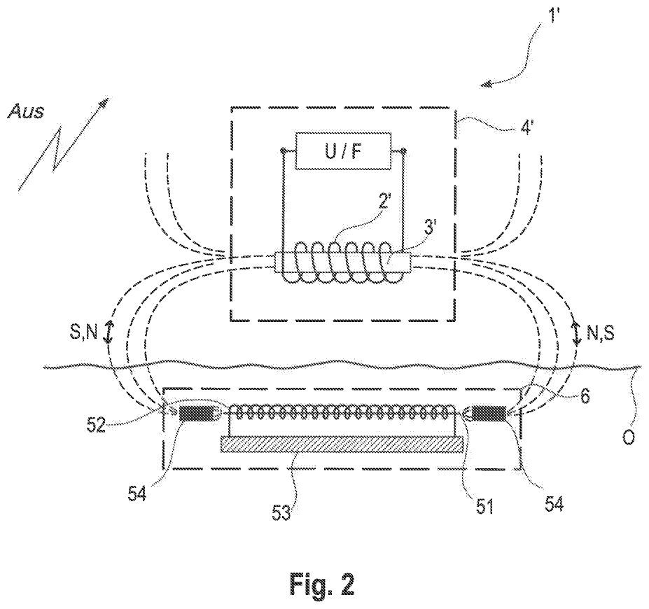

FIG. 2 shows a schematic illustration of a corresponding charging device;

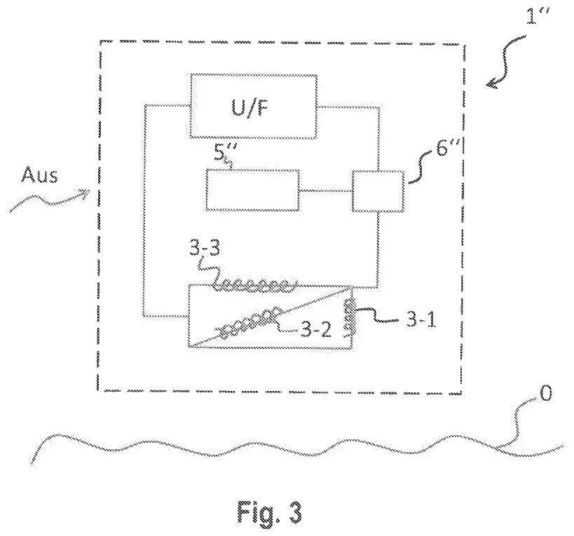

FIG. 3 shows an alternative configuration of the charging device shown in FIG. 2 with a plurality of coils.

FIG. 1 shows the schematic configuration of a pacemaker network A according to the invention, which comprises a plurality of pacemaker units. In the pacemaker network A of the invention, at least one of the pacemaker units acts as a master and the remaining pacemaker units respectively act as a slave, the behavior of which goes by the one of the master(s).

The pacemaker network A comprises, in context of the present preferred embodiment, the pacemaker unit 1 acting as the master and one pacemaker unit 1′ acting as the slave.

The pacemaker network A functions in the present preferred embodiment as a dual-chamber cardiac pacemaker, which is, in accordance with the intended use, fully implanted into the human body.

The pacemaker unit 1 acting as the master is preferably located inside the interior space of the atrium (Atrium) of the human heart or at its exterior, and is anchored there at a wall portion of the human heart (first body portion) by an electrode portion 2. The pacemaker unit 1′ acting as the slave, by contrast, is preferably located inside the interior space of a chamber (Ventricle) of the human heart or at its exterior, and is anchored there in a similar manner at a wall portion (second body portion) of the human heart via a corresponding electrode portion 2′.

The elements of the pacemaker unit 1 acting as the master and of the pacemaker unit 1′ acting as the slave, indicated by same reference signs are identical and will be described only once with reference to the pacemaker unit 1 acting as the master:

The electrode portion 2 is connected to an electronics assembly 3.

The electronics assembly 3 is configured to take the necessary functionality of the pacemaker unit 1 acting as the master. The electronics assembly 3 obtains an input signal Ein (body data), which allows the pacemaker unit 1 and the electronics assembly 3, respectively, to detect whether the monitored and controlled function needs to be stimulated and controlled, respectively. The electronics assembly obtains the input signal Ein (body data) either via the electrode portion 2 or via a separate monitoring electrode which is not shown.

If the electronics assembly 3, for example, detects that no own activity of the atrium (Atrium) is present after expiration of a certain time interval, the electronics assembly generates an impulse and stimulation impulse (current pulse and/or voltage pulse) respectively, which is output via the electrode portion 2 for stimulating the atrium (Atrium).

The electronics assembly is preferably configured to generate the impulse and to stimulate the atrium (Atrium) only in case of need, i.e. in case the electronics assembly 3 detects that an own activity is present until expiration of the predetermined time interval, the electronics assembly 3 behaves passively and does not generate the pulse.

The pacemaker unit 1 comprises an electric energy storage 4, for example an accumulator, which is connected to the electronics assembly 3, for supplying the electronics assembly 3. The electric energy storage 4, for example, Is a lithium ion accumulator which can be recharged. Another solution for an energy storage would be, for example, a capacitor with an extremely low self-discharge (e.g., gold cap).

Further, the pacemaker unit 1 acting as the master comprises a charging impulse generation portion 5, via which the energy storage 4 can be recharged. The charging impulse generation portion 5 allows for wireless charging of the energy storage 4.

The charging impulse generation portion 5 comprises, as essential elements, at least an impulse wire or Wiegand wire 51, which is axially surrounded by a coil 52 and around which the coil 52 is wound, respectively, and a charging electronics 53.

The impulse wire or Wiegand wire 51 forms a magnetization portion which can be influenced by a changing externally generated magnetic field. Preferably, the magnetization portion 51 may comprise a plurality of impulse wires and/or Wiegand wires, wherein each of the wires or a plurality of the wires may be surrounded by one or more coils.

The changing magnetic field is generated, for example, by a charging device, which will be explained below.

The magnetization portion 51 comprises evenly oriented magnetic domains which, when the magnetic field changes, start to remagnetize their polarities (to flip polarities) when a certain amplitude and field strength in the range of a few millitesla, respectively, is reached. From a physical aspect, this causes a remagnetization wave (Bloch wall) to run across the magnetization portion. In the literature, this event is also referred to as the big Barkhausen jump (großer Barkhausen-Sprung).

The size and speed of the remagnetization wave does not depend on the frequency (commutation frequency) with which the externally generated magnetic field changes. The remagnetization wave that runs across the magnetization portion generates a voltage impulse in the coil(s) 52 wound around the magnetization portion 51.

The voltage impulse is preferably processed by the charging electronics 53. The charging electronics 53 comprise for example a rectifier to rectify the voltage impulses of the coil(s), which are alternatingly generated with respective reverse polarities, and preferably a capacitor (for example also a gold cap) to temporarily store electrical energy.

The charging electronics 53 ultimately emit a charging impulse to the energy storage 4, which charges the same.

The electronics assembly 3 may preferably be designed to output a signal Aus, which indicates the quality of the charging impulse outputted by the charging electronics 53. The electronics assembly 3 detects, for example, the strength of the charging impulse and generates the signal Aus on its basis. The signal Aus is outputted, for example, as a low-frequency radio signal. The signal Aus is processed by the charging device still to be explained below.

The electronics assembly 3, the energy storage 4, and the charging impulse generation portion 5 are together accommodated in a casing 6 and are completely enclosed by the same. The casing 6 is preferably made from titanium or a corresponding alloy and is therefore extremely suitable for implantation into the human body because no rejection reactions occur and, as a metallic body, it keeps away high-frequency interfering fields. As metallic body, it may furthermore be used as the mass electrode that is necessary for the current impulse, which would not be possible with a glass body, for example. Forming the casing 6 as a glass body, however, is not excluded, and, in this case, the pacemaker unit 1 acting as the master includes a separate mass electrode, which is not shown in FIG. 1.

The described elements are also contained in the pacemaker unit 1′ acting as the slave and have an identical structure. I.e. the pacemaker unit 1′ acting as the slave obtains an input signal Ein via its electrodes portion 2 or a separate monitoring electrode, in order to detect whether an own activity of the chamber (ventricle) Is present or not. If necessary, the electronics assembly 3 of the pacemaker unit 1′ acting as the slave may generate the pulse and deliver it to the chamber (ventricle) which corresponds to a second body portion. Similar to the energy storage of the pacemaker unit 1 acting as the master, the energy storage of the pacemaker unit 1′ acting as the slave can be recharged in a wireless manner. For this purpose, the pacemaker unit 1′ acting as the slave comprises the already described charging impulse generation portion.

The pacemaker unit 1 acting as the master and the pacemaker unit 1′ acting as the slave forme together a pacemaker network, in which they interact with each other to realize the function of the dual-chamber cardiac pacemaker.

For this, the pacemaker unit 1 acting as the master includes a transmitting unit 31. The electronics assembly 3 is configured to transmit information via the transmitting unit 31, wherein the information has the informational content that the atrium has own activity or that the pulse was delivered to the atrium because no own activity of the atrium was detected by the electronics assembly. The transmitting unit 31 may work, for example, in accordance with the radio standard Bluetooth or low-energy Bluetooth; alternatively, the transmitting unit may also work in the range of low frequencies.

A difference of the operating modes of the pacemaker unit 1′ acting as the slave to the pacemaker unit 1 acting as the master exists in that the pacemaker unit 1′ acting as the slave normally does not function self-sufficiently but based on the transmitted information. For this purpose, the pacemaker unit 1′ acting as the slave comprises a receiving unit 31′ for receiving the transmitted information.

The electronics assembly 3 of the pacemaker unit 1′ acting as the slave starts, preferably based on the received information, a time measurement, which corresponds to an A/V-Interval (normal time interval between the own activity of the atrium and the ventricle) of a healthy heart. If the electronics assembly 3 of the pacemaker unit 1′ acting as the slave does not detect, based on the signal Ein, an own activity of the chamber until expiration of the A/V-interval, the electronics assembly generates a pulse and delivers it to the chamber (ventricle) via its electrode portion 2. Whereas the pacemaker unit 1′ detects, on the basis of said signal, an own activity of the chamber (ventricle), it behaves passively and, ergo, does not output an pulse.

The transmitting unit 31 of the pacemaker unit 1 acting as the master and the receiving unit 31′ of the pacemaker unit acting as the slave can both preferably have transmitting and receiving functions, and thus serve as an interface for programming the pacemaker unit(s).

FIG. 2 shows the schematic configuration of a charging device 1′ according to the invention, which is used to recharge the pacemaker units 1, 1′.

According to the invention, the invention generally uses an indirect induction method to recharge the energy storage 4, which means that the energy is not transmitted, as in the direct induction, directly and without delay from a primary coil to a secondary coil (transformer principle), but indirectly from a primary coil located in the charging device explained below, first to the energy-storing magnetization portion 51 and from there, with a delay, to the coil(s) 52 surrounding the magnetization portion 51.

The charging device 1′ comprises a coil (primary coil) 2′, for example, the current and/or voltage of which can be controlled in amplitude and frequency. Preferably, the col 2′ has a ferromagnetic core 3′, for example from ferrite, to amplify the field.

A casing 4′ accommodates the corresponding components of the charging device 1′. The casing 4′, if used as intended, is temporarily arranged near to or on a surface O of the human body so that the external magnetic field generated by the primary coil 2′ in the charging device reaches the magnetization portions 51 of the pacemaker units.

When the charging device 1′ is operated, it generates, by means of the primary coil 2′, an changing magnetic field which forms part of the electromagnetic field generated by the primary coil 2′. The generated changing magnetic field commutates with a certain commutation frequency and reaches the magnetization portions 51 of the pacemaker units. Each commutation leads, when a certain field strength is reached, to the initiation of the remagnetization wave, wherein the coil 52, which forms, in the indirect induction method mentioned above, the secondary coil, alternatingly generates positive and negative voltage impulses.

The voltage impulses are, as already explained, processed by the charging electronics 53 of the pacemaker units so that the charging electronics 53 ultimately output the charging impulse to the energy storage 4.

One essential point is, as already mentioned, that the contactless energy transmission is not based on the fact that the changing magnetic field, which is generated by the charging device 1′, is used directly for the voltage induction in the secondary col 52, but indirectly, in that the corresponding energy of the magnetic field is temporarily stored in the magnetization portion 51 and then released quite suddenly when the remagnetization wave is initiated, whereby the voltage impulse is generated in the secondary coil 52 by induction. For this reason, the commutation frequency may be adapted so that energy can be transmitted through the casing 6 made from metal without any problems as well.

The strength and/or commutation frequency of the magnetic field and the electromagnetic field, respectively, generated by the primary coil 2′, can be controlled in the charging device 1′ in order to adapt the recharging of the energy storage 4 of the pacemaker units to the specific location of the individual pacemaker units 1 in the body of the living being and to the necessary penetration depth, respectively, and in order to minimize the charge time. The controlling of the strength and/or commutation frequency of the magnetic field and the electromagnetic field, respectively, generated by the primary coil 2′ is performed in the charging device 1′ preferably on the basis of the signals Aus, which are output by the pacemaker units. To this purpose, the charging device 1′ has the corresponding receiving properties to receive the radio signals Aus.

Preferably, the pacemaker units comprise, in the longitudinal direction of the coil(s) 52 at end portions of its respective magnetization portion 51, magnetic collecting lenses 54 to bundle the changing magnetic field. The magnetic collecting lenses 54 may preferably have the form of a hollow cylinder into which the impulse wire/Wiegand wire or the impulse wires/Wiegand wires are inserted.

Alternatively or additionally to the magnetic collecting lenses 54, the respective casing 6 of the pacemaker units may be made of two assembled casing portions. The casing portions may be formed from ferromagnetic metals or be coated with such metals, wherein the orientation of the charging impulse generation portion 5 is selected within the casing 6 so that the casing portions function as additional or exclusive collecting lenses 54.

Per commutation of the changing external magnetic field, the remagnetization wave, which runs across the magnetization portion 51, is initiated, and ultimately one of the charging impulses to recharge the energy storage 4 is generated.

FIG. 3 shows an alternative embodiment of a charging device 1″ according to the invention.

The pacemaker units are arranged as shown in FIG. 2 on the basis of the pacemaker unit 1 acting as the master by way of example, but are not shown in FIG. 3 anymore.

The shown charging device 1″ differs from the one of FIG. 2 in that a plurality of coils 3-1, 3-2, 3-3, which respectively function as said primary coil, is provided. The charging device 1″ preferably comprises an electronics assembly 5″ and a multiplexer 6″. The electronics assembly 5″ is configured to control the multiplexer 6″ and to determine in this manner which or in which combination the coils 3-1, 3-2, 3-3 are used to generate the magnetic field. The controlling of the coils 3-1, 3-2, 3-3 is based on the (radio) signal(s) Aus from the pacemaker units, which indicate the quality of the charging impulse.

The coils 3-1, 3-2, 3-3 are spatially arranged differently, whereby the orientation of the magnetic field may be altered to improve and optimize the charging impulse.

Each of the coils 3-1, 3-2, 3-3 preferably comprises a core as shown in FIG. 2 and inserted in coil 2′.

For example, the functions of a multi-chamber cardiac pacemaker, such as the dual chamber cardiac pacemaker, may be realized by the pacemaker network of the invention. At the same time, the electrode portions 2 are formed very short and do not require, for example, a plastic insulation, which could lead to health problems.

In addition, the pacemaker units, due to their wireless recharging, easily can be made so small that they can be arranged directly in or on the heart. The casing 6 of the pacemaker units 1, 1′ has, for example, a volume of less than/equal to 1 cm³ and a weight of a few grams, for example of 0.7 g. Thus, the casing 6 has little mass to be accelerated.

1. Wireless pacemaker network for implantation in a body of a living being and for controlling a bodily function, wherein the pacemaker network comprises:
an electronic pacemaker unit (1) that acts as a master in the pacemaker network, comprising
an electrode portion (2) that is, according to its intended purpose, to be attached to/arranged at a first body portion, and
an electronics assembly that is adapted to monitor a function of the first body portion preferable via the electrode portion and/or to generate a pulse, in particular a voltage pulse, and to output it via the electrode portion to the first body portion; and
an electronic pacemaker unit (1′) that acts as a slave in the pacemaker network, comprising
an electrode portion (2′) that is, according to its intended purpose, to be attached to/arranged at a second body portion, and
an electronics assembly connected with the electrode portion, which is configured to generate a pulse, in particular a voltage pulse, and to output it to the second body portion via the electrode portion; wherein
the pacemaker unit (1) acting as the master and pacemaker unit (1′) acting as the slave are configured to interact wirelessly for controlling the bodily function, by the pacemaker unit acting as the slave
(i) to obtain information on the function of the first body portion from the pacemaker unit (1) acting as the master and/or on the delivery of the pulse to the first body portion, and
(ii) to determine, based on the information, whether and/or when the pulse is delivered to the second body portion;
wherein pacemaker unit (1) acting as the master and/or the pacemaker unit (1′) acting as the slave comprises:
an energy storage to supply the corresponding electronics assembly with electrical energy, which can be recharged with electrical energy after discharge; and
a charging impulse generation portion electrically connected to the energy storage, which is configured to be able to emit a charging impulse to the energy storage for recharging the energy storage;
wherein
the charging impulse generation portion comprises a magnetization portion with oriented magnetic domains, which can be contactlessly influenced by a changing magnetic field so that, when a certain field strength is reached, a remagnetization wave, caused by the continuously reversing magnetic domains, occurs in the magnetization portion, which runs across the magnetization portion and leads to the generation of the charging impulse.

2. Pacemaker network according to claim 1, wherein
the pacemaker unit (1) acting as the master has a transmitting unit and is configured to transmit the information via the transmitting unit; and
the pacemaker unit (1′) acting as the slave has a receiving unit and is configured to obtain the information by receiving the information transmitted by the transmitting unit via the receiving unit.

3. Pacemaker network according to claim 1, wherein
the pacemaker unit (1′) acting as the slave has a detection unit and is adapted to obtain the information by detecting the pulse delivered by the pacemaker unit (1) acting as the master via the detection unit.

4. Pacemaker network according to claim 1, wherein the charging impulse generation portion comprises at least one coil which is spatially arranged to the magnetization portion, preferably wound around the magnetization portion surrounding it axially, so that it generates a voltage pulse, which leads to the charging impulse, when the remagnetization wave occurs.

5. Pacemaker network according to claim 4, wherein the magnetization portion is formed by mechanical machining so that the magnetic domains of the magnetization portion are equally oriented.

6. Pacemaker network according to claim 5, wherein the magnetization portion comprises a magnetically hard shell area which encloses a magnetically soft core area.

7. Pacemaker network according to claim 1, wherein the magnetization portion is at least an impulse wire or a Wiegand wire.

8. Pacemaker network according to claim 7, wherein the magnetization portion comprises a plurality of impulse wires or a plurality of Wiegand wires or a combination of at least one impulse wire and one Wiegand wire.

9. Pacemaker network according to claim 8, wherein
the coil is wound around the plurality or the combination of wires, or
several coils are provided which are each wound around at least one of the wires.

10. Pacemaker network according to claim 1, wherein the charging impulse generation portion comprises, in a direction in which the at least one coil is wound, a magnetic collecting lens at at least one end portion of the magnetization portion for bundling and guidance of the changing magnetic field to the magnetization portion.

11. Pacemaker network according to claim 1, wherein the electronics assembly of the pacemaker unit (1) acting as the master and/or of the pacemaker unit (1′) acting as the slave is fully surrounded, together with the respective energy storage and the respective charging impulse generation portion, by a sleeve or a casing which is formed of a material that is not rejected by the body of the living being.

12. Pacemaker network according to claim 11, wherein the material is a preferably non-ferromagnetic metal, in particular titanium, or a metal alloy comprising titanium in particular.

13. Pacemaker network according to claim 1, wherein the pacemaker unit (1) acting as the master and/or the pacemaker unit (1′) acting as the slave are designed such that the respective electronics assembly and/or the respective charging impulse generation portion apart from the magnetization portion and, if preferably provided, the at least one magnetic collecting lens, does not comprise elements made of ferromagnetic materials.

14. Pacemaker network according to claim 1, wherein the electronics assembly of pacemaker unit (1) acting as the master and/or of the pacemaker unit (1′) acting as the slave is configured to transmit a signal indicative of the quality of the charging impulse.

15. Pacemaker network according to claim 1, wherein the energy storage of the pacemaker unit (1) acting as the master and/or of the pacemaker unit (1′) acting as the slave, is an accumulator, such as a lithium-ion accumulator.

16. Pacemaker network according to claim 1, wherein the energy storage of the pacemaker unit (1) acting as the master and/or of the pacemaker unit (1′) acting as the slave is a capacitor with low self-discharge.

17. Pacemaker network according to claim 10, wherein the at least one magnetic collecting lens is formed of a ferromagnetic metal which bundles the magnetic field for the magnetization portion.

18. Charging device for a pacemaker network, wherein
the charging device is configured to generate a magnetic field that changes with a commutation frequency and preferably amplitude, and
the charging device, when used as intended, is arranged on a body surface of the living being or close to the body surface of the living being so that the magnetic field penetrates the body and the implanted pacemaker unit (1) acting as the master and/or the pacemaker unit (1′) acting as the slave of a pacemaker network according to claim 1, to influence the corresponding charging impulse generation portion.

19. Charging device according to claim 11, wherein the commutation frequency is in a range from
X to 10 kHz, wherein X>0 and X>=0.1 kHz, 0.2 kHz, 0.3 kHz, . . . , 4.9 kHz, . . . , or 9.9 kHz.

20. Charging device according to claim 18, wherein a plurality of coils is provided for the generation of the changing magnetic field, which coils can be controlled accordingly on the basis of the signal(s) indicating the quality of the charging impulse(s), for an optimization of the charging impulse(s).
